(19) United States
(12) Patent Application Publication
(10) Pub. No.: US 2013/0190675 A1
(43) Pub. Date: Jul. 25, 2013

(54) Methods and Devices for Treating Alzheimer's Disease

(71) Applicant: Aaron Sandoski, Lincoln, MA (US)

(72) Inventor: Aaron Sandoski, Lincoln, MA (US)

(21) Appl. No.: 13/750,344

(22) Filed: Jan. 25, 2013

Related U.S. Application Data
(60) Provisional application No. 61/590,367, filed on Jan. 25, 2012.

(51) Int. Cl. A61F 5/00; A61B 17/11

(52) U.S. Cl. CPC A61F 5/0076; A61B 17/1114
USPC 604/8

(57) ABSTRACT

A method and apparatus of treating Alzheimer's disease in an individual. In one embodiment, the method includes bypassing a portion of the gastrointestinal tract of the individual. In another embodiment, the method includes modifying insulin levels or insulin resistance in the brain of an individual with Alzheimer's disease or predisposed to Alzheimer's disease. In still yet another embodiment, the bypass causes the individual's serum levels of GLP-1 protein to increase.

### RELATED APPLICATIONS

This application claims priority to U.S. Provisional Application 61/590,367, filed Jan. 25, 2012, the contents of which are herein incorporated by reference in their entirety.

### FIELD OF THE INVENTION

The invention relates to the field of gastrointestinal devices and more specifically to gastrointestinal devices modulating the production and utilization of insulin.

### BACKGROUND

Alzheimer's disease is a common form of age-related dementia that affects millions of people. Alzheimer's disease is characterized by the formation of amyloid plaques in the brain, which arise from the deposition of insoluble amyloid-beta peptides.

While the etiology of Alzheimer's disease remains unclear, it is believed the presence of amyloid plaques results in neuronal death, which may contribute to the gradual loss of brain functions observed in Alzheimer's patients. However, studies have shown that amyloid plaque can be found in people who do not have Alzheimer's disease. Thus, the presence of amyloid plaques in Alzheimer's patients is believed to be a correlation rather than a causative agent.

There is some speculation that a reduction in cerebral blood flow contributes to Alzheimer's disease. However, it has been found that reductions in cerebral blood flow precede some forms of dementia (e.g., vascular and ischemic) but not Alzheimer's disease. Therefore, reduced cerebral blood flow likely is a result of Alzheimer's disease rather than a cause.

Alzheimer's patients have a higher propensity to have type II diabetes than a matched control group without Alzheimer's disease. Although there is a correlation between the two diseases, a majority of Alzheimer's patients do not have diabetes. Type I and type II diabetes are not viewed as the cause of Alzheimer's disease, although underlying mechanisms may be similar or related. In addition, in normal individuals, the amount of insulin in the brain is much less than the systemic insulin circulating the body. Insulin can cross the blood-brain barrier, but it is not known whether insulin abnormalities observed in the brains of Alzheimer's patients stem from abnormal systemic insulin levels or from local problems in the brain and the organ's ability to produce and/or use insulin. It is possible that changes in the body's insulin levels too small to manifest as type II diabetes may still be large enough to affect the much smaller levels of insulin in the brain. Recently, it has been shown that people with Alzheimer's, but without diabetes, show brain insulin resistance.

Another possible cause of Alzheimer's disease is abnormal brain levels of insulin. The hippocampus is the region of the brain with the greatest number of insulin receptors. The hippocampus also is the core region responsible for memory and it is the earliest region of the brain to be affected by Alzheimer's disease. In addition, there is much evidence that increasing or decreasing the levels of insulin in the brain can have an effect on memory. Researchers have showed positive results for slowing the progression of Alzheimer's disease in humans by directly administering insulin to the brain via absorption in the nasal cavity or intranasal injection through the olfactory bulb, thereby bypassing the blood-brain barrier. Some have termed Alzheimer's disease as type III diabetes because of the evidence linking reductions in brain insulin or insulin resistance to Alzheimer's disease.

In addition to direct injection of insulin to the brain, some researchers have explored drugs and hormones used in type II diabetes as a new therapeutic approach to treating Alzheimer's. One group of compounds that shows positive results in animal testing are compounds that target production of GLP-1. GLP-1 is a hormone that plays a key role in type II diabetes because of its ability to regulate the production of insulin and its effects on insulin resistance in the body.

Bariatric surgery that bypasses a large portion of the intestines (e.g., Rouz-En-Y) has been shown to significantly elevate levels of GLP-1 post-surgery. These changes occur immediately after bypass surgery and are not dependent on weight loss. The altered gastrointestinal tract causes hormone changes in the body. A leading theory is that raw food in the lower gastrointestinal tract is a signaling mechanism for the body to produce GLP-1, which then increases insulin levels. By bypassing a portion of the gastrointestinal tract, more raw food enters the lower gastrointestinal tract and a greater trigger is created thus generating more GLP-1. In addition to increasing GLP-1 production, type II diabetics having bypass surgery also have a high likelihood of eliminating their type II diabetes immediately post-operative. Additional explanations for the immediate resolution of type II diabetes include hormone changes resulting from modified nutrient patterns in the duodenum and reduction in inflammation. It is likely that all of these factors have an effect with the increase in GLP-1 being the dominant effect. One study published in July 2012 looked at biomarkers for Alzheimer's at six months following bariatric surgery. The researchers found an over 30% reduction in expression for amyloid precursor protein, the precursor of beta-amyloid, and a significant reduction in the expression of other Alzheimer's-related genes. The researchers attributed the changes to a reduction in obesity (BMI went from 52.1 to 40.4 kg/m²) and diabetes (HbA1c went from 7.9 to 6.3%).

Medical devices that try to induce the same obesity and diabetes changes as bariatric surgery are being tested in clinical trials by companies such as GI Dynamics and Valentx. The devices attempt to replicate the surgical results by using a liner in the GI tract to allow food to bypass a portion of the GI tract. These devices have shown positive results for diabetes control when the devices are used.

What is needed is a medical device that will slow or prevent Alzheimer's disease. The present invention addresses this need.

### SUMMARY OF THE INVENTION

In one aspect, the invention relates to a method and apparatus of treating Alzheimer's disease in an individual. In one embodiment, the method includes bypassing a portion of the gastrointestinal tract of the individual. In another embodiment, the method includes modifying insulin levels or insulin resistance in the brain of an individual with Alzheimer's disease or predisposed to Alzheimer's disease. In still yet another embodiment, the bypass causes the individual's serum levels of GLP-1 protein to increase.

In another embodiment, the bypassing comprises inserting a gastrointestinal tube that lines the portion of the gastrointestinal tract, the gastrointestinal tube reducing nutrient absorption. In yet another embodiment, the portion of the gastrointestinal tract is selected from the group consisting of: stomach, stoma, duodenum, jejunum, ileum, and combinations thereof. In still yet another embodiment, the bypassing comprises: forming a surgical anastomosis by suturing a distal portion of the gastrointestinal tract to a proximal portion of the gastrointestinal tract; or implanting an anastomosis device by surgical or gastrointestinal intervention. In one embodiment, the bypassing includes surgically reducing the length or volume of the stomach, duodenum, or small intestines.

### BRIEF DESCRIPTION OF THE DRAWINGS

FIG. 1 is a diagram showing a gastrointestinal liner, in accordance with an illustrative embodiment; and

FIG. 2 is a diagram of an anastomosis being used to perform the function of the invention.

### DETAILED DESCRIPTION

The invention provides methods for treating Alzheimer's disease. The methods include bypassing a large portion of the gastrointestinal tract to reduce insulin resistance in the brain. Unlike bariatric surgery and the medical devices trying to replicate bariatric procedures less invasively, this treatment for Alzheimer's does not specifically involve the objective of weight loss and/or type II diabetes resolution, although these may be secondary objectives for patients with high BMIs and type II diabetes. Bypassing the gastrointestinal tract or a portion of the gastrointestinal tract induces bodily changes, including increased GLP-1 production, which in turn increases insulin production and/or sensitivity. The present invention provides therapeutic benefit by increasing brain insulin levels and/or compensating for insulin resistance in the brain. Methods for accomplishing gastrointestinal bypass include surgery, a device that lines part of the gastrointestinal tract, and/or a device that creates an anastomosis from a distal part of the gastrointestinal tract to a proximal part of the gastrointestinal tract.

The invention provides a new approach to treating Alzheimer's disease. The approach includes bypassing a portion of the gastrointestinal tract in order to stimulate GLP-1 production, which increases insulin production and/or compensates for insulin resistance in the brain. Unlike Roux-En-Y surgery for bariatrics, which involves bypassing the duodenum, the treatment of Alzheimer's can involve bypassing any portion of the gastrointestinal tract since weight loss is not an objective of the bypass. Instead, one objective is to deliver raw or partially digested food to the lower gastrointestinal tract. Stimulating the lower GI tract to produce GLP-1 in order to affect brain insulin sensitivity requires additional raw food to enter the lower intestines. A device that routes some food, but not necessarily all food, to the lower GI tract would have a beneficial effect on GLP-1 production, yet not have the negative effects of malabsorption and other complications found in bariatric surgery and device alternatives, since all of these procedures divert all contents in the bowel.

Non-limiting methods for creating an adequate bypass include surgery, a device that lines part of the gastrointestinal tract, and/or a device that creates an anastomosis from a distal part of the gastrointestinal tract to a proximal part of the gastrointestinal tract.

A gastrointestinal liner reduces nutrient absorption and it also reduces contact between ingested food and digestive enzymes, resulting in more raw food reaching the lower gastrointestinal tract. A gastrointestinal liner for use in treating Alzheimer's disease can be implanted and positioned to bypass any portion of the gastrointestinal tract. For example, the gastrointestinal liner can bypass part of the stomach (e.g., stoma) and/or part of the small intestine (e.g., part of the duodenum, jejunum, or ileum).

Surgical methods can be employed to excise part of the gastrointestinal tract to reduce the length and/or volume of the proximal, or upper, gastrointestinal tract. Surgical methods also can be used to create an anastomosis between distal and proximal portions of the gastrointestinal tract, such as a Roux-En-Y procedure, where a distal part of the gastrointestinal tract (e.g., the ileum) is sutured to a proximal part of the gastrointestinal tract (e.g., the stomach).

In addition, anastomosis devices can be implanted to bypass a portion of the gastrointestinal tract. Anastomosis devices can be implanted using invasive surgical procedures and/or using non-invasive gastrointestinal interventions. Anastomosis devices, such as anastomosis rings, can be implanted.

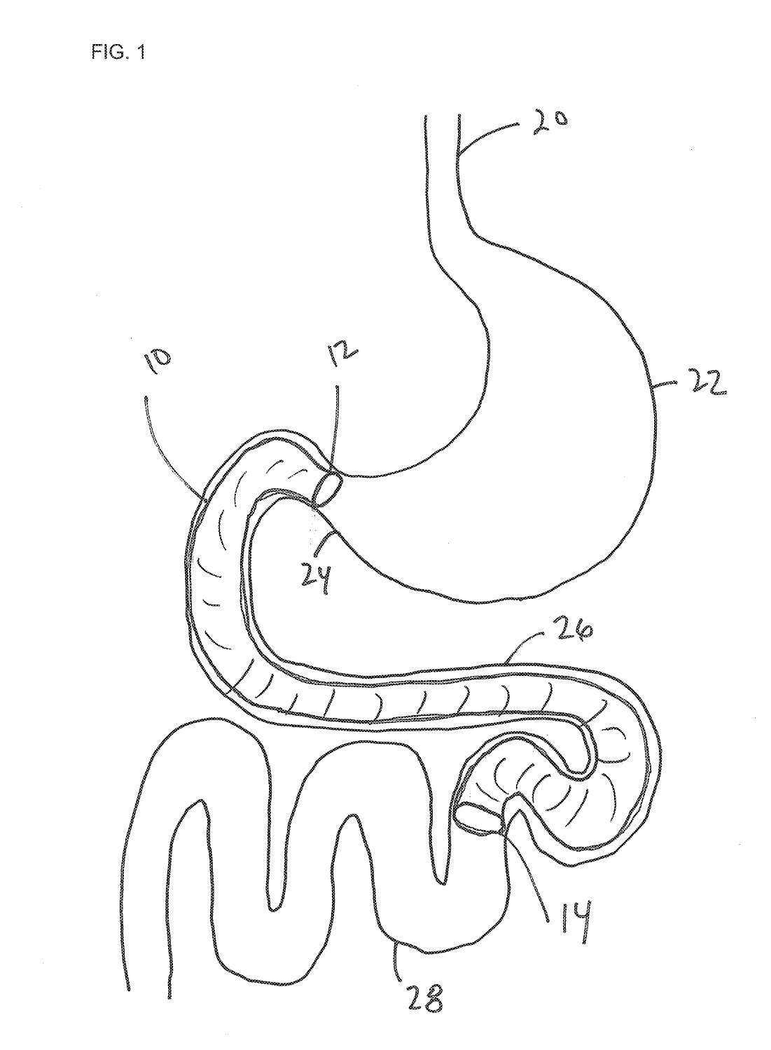

FIG. 1 shows a gastrointestinal liner deployed in a portion of the gastrointestinal tract. The gastrointestinal liner 10 can be inserted by non-invasive gastrointestinal delivery via the esophagus 20 and stomach 22, for example. Gastrointestinal liners and delivery methods are well known in the art. Briefly, a first end 12 of the gastrointestinal liner 10 is positioned at a proximal location of the gastrointestinal tract, such as the stoma 24. A second end 14 of the gastrointestinal liner 10 is positioned distal to the first end 12, such as in the jejunum 28 of the small intestine. As a result, the gastrointestinal liner shown in FIG. 1 bypasses the duodenum 26 and a portion of the jejunum 28. As will be appreciated, the length and position of the gastrointestinal liner will determine which portions of the gastrointestinal tract are bypassed.

For example, if a patient presents with Alzheimer's disease a gastrointestinal liner can be implanted in the patient to bypass the proximal, or upper, portion of the small intestine from the duodenum to the jejunum. It is anticipated that gastrointestinal bypass will result in reestablishing normal insulin levels in the brain and that normal brain insulin activity will delay or mitigate Alzheimer's disease progression.

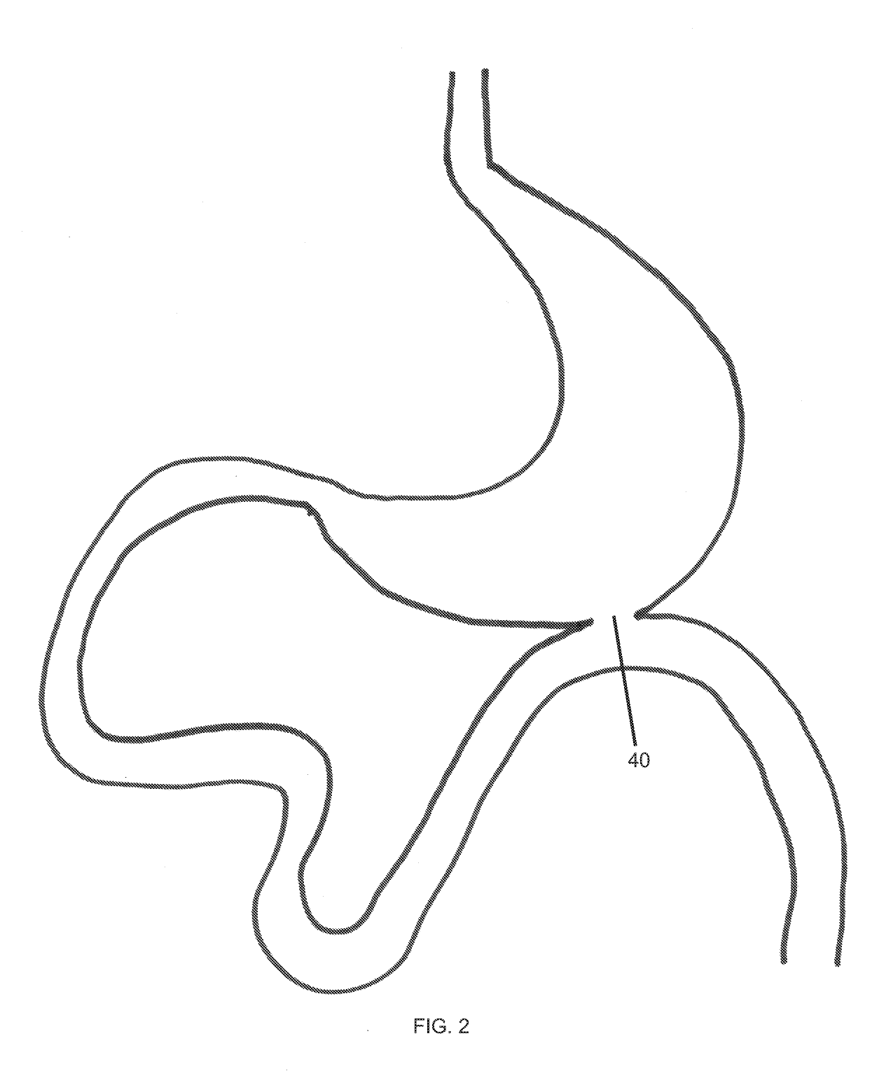

FIG. 2 is a diagram of an anastomosis 40 in the gastrointestinal tract that functions to partially bypass only a portion of the gastrointestinal tract. Using this surgical technique, more raw food will also reach the lower gastrointestinal tract, again resulting in an increase in GLP-1.

What is claimed is:

1. A method of treating Alzheimer's disease in an individual, the method comprising bypassing a portion of the gastrointestinal tract of the individual.

2. A method of modifying insulin levels or insulin resistance in the brain of an individual with Alzheimer's disease or predisposed to Alzheimer's disease, the method comprising bypassing a portion of the gastrointestinal tract of the individual.

3. The method of claim 1 or 2, whereby the bypass causes the individual's serum levels of GLP-1 protein to increase.

4. The method of claim 1 or 2, wherein the bypassing comprises inserting a gastrointestinal tube that lines the portion of the gastrointestinal tract, the gastrointestinal tube reducing nutrient absorption.

5. The method of claim 1 or 2, wherein the portion of the gastrointestinal tract is selected from the group consisting of: stomach, stoma, duodenum, jejunum, ileum, and combinations thereof.

6. The method of claim 1 or 2, wherein the bypassing comprises:
(i) forming a surgical anastomosis by suturing a distal portion of the gastrointestinal tract to a proximal portion of the gastrointestinal tract; or
(ii) implanting an anastomosis device by surgical or gastrointestinal intervention.

7. The method of claim 1 or 2, wherein the bypassing comprises surgically reducing the length or volume of the stomach, duodenum, or small intestines.
